(19) United States
(12) Patent Application Publication
(10) Pub. No.: US 2015/0351659 A1
(43) Pub. Date: Dec. 10, 2015

(54) MAGNETIC RESONANCEIMAGING APPARATUS AND MEASUREMENT METHOD THEREOF

(71) Applicant: (HITACHI MEDICAL CORPORATION), Chiyoda-ku, Tokyo (JP)

(72) Inventor: Yoshiyuki Kunugi, Tokyo (JP)

(21) Appl. No.: 14/760,522

(22) PCT Filed: Feb. 4, 2014
(86) PCT No.: PCT/JP2014/052527

§ 371 Date: Jul. 13, 2015

(30) Foreign Application Priority Data
Feb. 12, 2013 (JP) 2013-024991

(51) Int. Cl. A61B 5/055

(52) U.S. Cl. CPC A61B 5/055; A61B 2576/00

(57) ABSTRACT

There is provided a magnetic resonance measuring apparatus for acquiring spectral data with reduced influence of contamination signals from the outside of a volume of interest. For this, a magnetic resonance imaging apparatus according to the present invention acquires a first echo signal generated from an object based on a gradient magnetic field having one polarity generated by a gradient magnetic field generation unit, acquires a second echo signal generated from the object based on a gradient magnetic field having the other polarity, which is a polarity opposite to the one polarity, generated by the gradient magnetic field generation unit, and creates a graph indicating the state of metabolites using both of the first echo signal and the second echo signal.

### TECHNICAL FIELD

The present invention relates to a magnetic resonance imaging apparatus and a measurement method thereof.

### BACKGROUND ART

Currently, an image that reflects the density distribution of hydrogen nuclei contained mainly in water molecules in an object is acquired by magnetic resonance imaging (hereinafter, referred to as “MRI”) that is widespread.

In addition to the MRI, there is a method called magnetic resonance spectroscopy (hereinafter, referred to as “MRS”) in which magnetic resonance signals for each of molecules chemically bonded to each other are separated from each other based on a resonance frequency difference (hereinafter, referred to as a chemical shift) due to differences in the chemical bond of various molecules containing hydrogen nuclei.

In addition, a method of acquiring the spectra of a number of regions (pixels) simultaneously and performing imaging for each molecule is called magnetic resonance spectroscopic imaging or chemical shift imaging. Hereinafter, these are collectively referred to as “MRSI”. By using the MRSI, it is possible to grasp the concentration distribution of each metabolite visually.

In the measurement of MRS or MRSI, a method is generally used in which slices perpendicular to each other are selectively excited by applying three high-frequency pulses (RF pulses) and signals are acquired from the volume formed by the crossing of the slices. As an irradiation frequency used in the irradiation of the RF pulses, a frequency in the range of the resonance frequency of water or a frequency in the range of the resonance frequency of the metabolite to be measured (for example, in the head, inositol, choline, creatine, glutamine, glutamic acid, GABA, NAA, and lactic acid) is generally used. If the irradiation frequency and the resonance frequency of the metabolite are different, resonance occurs at a frequency shifted from the irradiation frequency due to the chemical shift. As a result, an MR signal is generated from a region shifted from a volume of interest VOI set on the positioning image.

For the shift of the excitation position of each metabolite, the amount of shift of the excitation position is determined by the strength of the slice selection gradient magnetic field, and the shift direction is determined by the polarity of the slice selection gradient magnetic field. In addition, the shift of the excitation position occurs according to each RF. In general, however, the user is not notified of such information in many cases. In this case, signals are acquired in a region that is not intended by the user. As a result, contamination signals are displayed in the spectral data.

PTL 1 discloses an MRSI apparatus that reduces the chemical shift error by suppressing the generation of signals from the outside of the volume of interest by applying a saturation pulse with very high selectivity to the outside of the volume of interest.

### CITATION LIST

### Patent Literature

[PTL 1] Japanese Patent No. 4383568

### SUMMARY OF INVENTION

### Technical Problem

In the MRSI apparatus disclosed in PTL 1, a saturation pulse with very high selectivity is applied to the outside of the volume of interest. However, since the saturation pulse is applied while being aware of the direction of the excitation position shift for each metabolite due to the chemical shift, time and effort have been required. For this reason, a magnetic resonance measuring apparatus for acquiring the spectral data with the reduced influence of contamination signals from the outside of the volume of interest using an easier method has been demanded.

It is an object of the present invention to provide a magnetic resonance imaging apparatus capable of easily reducing the influence of contamination signals from a region outside the volume of interest in the acquisition of spectral data.

In order to solve the problems described above, there is provided a magnetic resonance imaging apparatus, including: a gradient magnetic field generation unit configured to generate a gradient magnetic field for the object; an echo signal receiving unit configured to receive an echo signal from the object; and a control information processing unit. The control information processing unit acquires a first echo signal generated from the object based on a gradient magnetic field having one polarity generated by the gradient magnetic field generation unit, acquires a second echo signal generated from the object based on a gradient magnetic field having the other polarity, which is a polarity opposite to the one polarity, generated by the gradient magnetic field generation unit, and creates information indicating the state of the metabolite using both of the first and second echo signals.

### Advantageous Effects of Invention

According to the present invention, it is possible to provide a magnetic resonance imaging apparatus capable of easily reducing the influence of contamination signals from the outside of the volume of interest in the acquisition of spectral data.

### BRIEF DESCRIPTION OF DRAWINGS

FIG. 1 is a block diagram showing the overall configuration of an MRI apparatus according to an embodiment of the present invention.

FIG. 2 is a functional block diagram showing the main function of a control information processing system.

FIG. 3 is an explanatory diagram showing an example of the pulse sequence used in the MRSI measurement.

FIG. 4 is an explanatory diagram showing an excitation region of each RF pulse in the head of a person when the pulse sequence shown in FIG. 3 is used.

FIG. 5 is a diagram showing the position shift of an excitation region due to chemical shift for a region excited by RF1.

FIG. 6 is a diagram showing the position shift of an excitation region due to chemical shift for a region excited by each RF pulse.

FIG. 7A is a flowchart for explaining the flow of the process in a first embodiment.

FIG. 7B is a flowchart for explaining the flow of the process in the first embodiment.

FIG. 8 is a diagram showing the position shift of an excitation region due to chemical shift when the polarity of the slice selection gradient magnetic field is inverted, which shows the characteristics of the first embodiment.

FIG. 9 is a diagram of a signal strength spectrum showing the result in the first embodiment.

FIG. 10 is a diagram for explaining the flow of the process in a second embodiment.

FIG. 11 is a diagram for explaining the flow of the process in a third embodiment.

FIG. 12 is a diagram showing a set volume of interest and an excitation region of RF1, which shows the characteristics of the third embodiment.

FIG. 13 is a diagram for explaining the flow of the process in a fourth embodiment.

FIG. 14 is a diagram showing the region setting of a Presat pulse that covers an excitation region outside the volume of interest, which shows the characteristics of the fourth embodiment.

### DESCRIPTION OF EMBODIMENTS

Hereinafter, a form (hereinafter, referred to as an embodiment) for carrying out the present invention will be described with reference to the diagrams. In addition, in all diagrams for explaining the embodiments according to the invention, components having the same functions are denoted by the same reference numerals, and repeated explanation thereof will be omitted.

### First Embodiment

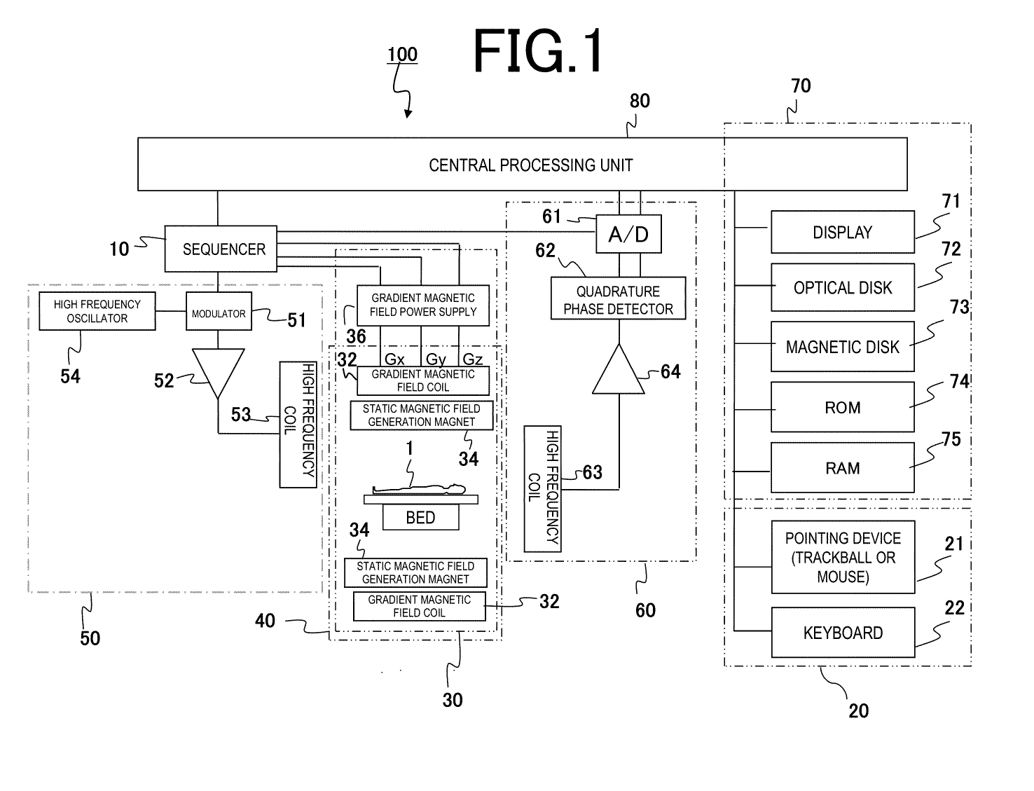

FIG. 1 is a block diagram showing the overview of an MRI apparatus 100 that is an embodiment of the present invention. The MRI apparatus 100 acquires a tomographic image of an examination part of an object or functional information of the body using a nuclear magnetic resonance (hereinafter, abbreviated as “NMR”) phenomenon. As shown in FIG. 1, the MRI apparatus 100 is configured to include a static magnetic field generation system 40 that generates a static magnetic field, a gradient magnetic field generation system 30 that generates a gradient magnetic field, a signal transmission system 50 that transmits an RF signal, a signal receiving system 60 that receives a signal based on the NMR phenomenon, a control information processing system 70 that processes the received signal and performs various kinds of processing or control, a sequencer 10, a central processing unit (hereinafter, referred to as a CPU) 80, and an operating unit 20 that is operated by an operator. In addition, although not clear in the block diagram of FIG. 1, the control information processing system 70 includes a CPU 80. The CPU 80 is used to process the signal received by the signal receiving system 60, and the CPU 80 is used for various kinds of control or information processing performed by the control information processing system 70.

The static magnetic field generation system 40 includes a static magnetic field generation magnet 34 disposed around the measurement space into which an object 1 is inserted. The static magnetic field generation magnet 34 may be of a permanent magnet type using a permanent magnet, a normal conduction type using a normal conducting magnet, or a superconducting type using a superconducting electromagnet, and the present invention is effective for any type. In a vertical magnetic field method used when the MRI apparatus 100 is an open type MRI apparatus, the static magnetic field generation system 40 generates a uniform static magnetic field in the measurement space, into which the object 1 is inserted, in a direction perpendicular to the body axis. Ina horizontal magnetic field method used when the MRI apparatus 100 is a tunnel type MRI apparatus, the static magnetic field generation system 40 generates a uniform static magnetic field in the body axis direction.

The gradient magnetic field generation system 30 is configured to include a gradient magnetic field coil 32 that applies a gradient magnetic field in three axial directions of X, Y, and Z, which are the coordinate system, that is, the stationary coordinate system of the MRI apparatus, and a gradient magnetic field power supply 36 to drive each gradient magnetic field coil 32. Gradient magnetic fields Gx, Gy, and Gz are applied in the three axial directions of X, Y, and Z by driving the gradient magnetic field power supply 36 of each coil according to the command from the sequencer 10.

At the time of MRI imaging, a slice direction gradient magnetic field pulse (hereinafter, referred to as a slice selection gradient magnetic field) is applied in a direction perpendicular to the slice plane, that is, the imaging cross-section in order to set the slice plane for the object 1, and a phase encoding direction gradient magnetic field pulse Gp and a frequency encoding direction gradient magnetic field pulse Gf are applied in two remaining directions, which are perpendicular to the slice plane and are also perpendicular to each other, in order to encode position information in each direction in an echo signal (Sig).

At the time of MRS or MRSI measurement, a volume of interest for the object 1 is set by applying the slice direction gradient magnetic fields in a direction in which the slice direction gradient magnetic fields are perpendicular to each other, and the echo signal (Sig) is obtained without performing frequency encoding. In the MRSI measurement, the position information is encoded by applying the phase encoding direction gradient magnetic field pulses in two directions or three directions.

The sequencer 10 is a control unit for repeatedly applying RF pulses and gradient magnetic field pulses according to a predetermined pulse sequence, and operates under the control of the CPU 80 and transmits various commands, which are required to collect the data of a tomographic image of the object 1, to the signal transmission system 50, the gradient magnetic field generation system 30, and the signal receiving system 60.

The signal transmission system 50 emits an RF pulse to the object 1 in order to cause nuclear magnetic resonance in the nuclear spins of atoms that form the body tissue of the object 1, and includes a high frequency oscillator 54, a modulator 51, a high frequency amplifier 52, and a transmission coil, that is, a high frequency coil 53 on the transmission side. An RF pulse output from the high frequency oscillator 54 is amplitude-modulated by the modulator 51 at a timing according to the command from the sequencer 10, and the amplitude-modulated RF pulse is amplified by the high frequency amplifier 52. Then, the amplified RF pulse is supplied to the high frequency coil 53 disposed close to the object 1, and is emitted to the object 1 from the high frequency coil 53.

The signal receiving system 60 has a function of detecting an NMR signal, which is the echo signal (Sig) emitted by the nuclear magnetic resonance of the nuclear spins that form the body tissue of the object 1, and includes a high frequency coil 63 on the receiving side that is a receiving coil, a signal amplifier 64, a quadrature phase detector 62, and an A/D converter 61 that converts an analog signal into a digital signal. The NMR signal of the response of the object 1 induced by the electromagnetic waves emitted from the high frequency coil on the transmission side 53 is detected by the high frequency coil 63 disposed close to the object 1, and is amplified by the signal amplifier 64. Then, at a timing according to the command from the sequencer 10, the amplified NMR signal is divided into two signals perpendicular to each other by the quadrature phase detector 62, and each of the signals is converted into a digital amount by the A/D converter 61 and is transmitted to the control information processing system 70 so as to be processed.

The control information processing system 70 performs various kinds of data processing, display of processing results, storage of processing results and required information, and the like, and includes an external storage device, such as an optical disk 72, a magnetic disk 73, a ROM 74, and a RAM 75, and a display 71, such as a CRT or a liquid crystal display device. The control information processing system 70 further includes the CPU 80 that performs various kinds of processing or control. When data from the signal receiving system 60 is input to the CPU 80, the CPU 80 performs processing, such as signal processing and image reconstruction, and displays a tomographic image of the object 1, which is the result, on the display 71 and records the tomographic image on the magnetic disk 73 or the like of the external storage device.

The operating unit 20 is used to input control information required for the processing performed in the control information processing system 70 of the MRI apparatus, and includes a pointing device 21 and a keyboard 22. The pointing device 21 is a trackball, a mouse, or a touch panel, for example, and is used to perform the input of the positional relationship with respect to the display content displayed on the display 71 or the selection operation on the display content. The operating unit 20 is disposed close to the display 71, so that the operator can control various kinds of processing of the MRI apparatus interactively through the operating unit 20 while watching the display 71.

In FIG. 1, the high frequency coil 53 on the transmission side and the gradient magnetic field coil 32 are provided in the static magnetic field space of the static magnetic field generation system 40, into which the object 1 is inserted, so as to face the object 1 in the vertical magnetic field method and so as to surround the object 1 in the horizontal magnetic field method. In addition, the high frequency coil 63 on the receiving side is provided so as to face the object 1 or surround the object 1.

Currently, nuclides imaged by the MRI apparatus, which are widely used clinically, are a hydrogen nucleus (hereinafter, referred to as proton) that is a main component material of the object. The shapes, functions, or bioinformation of the head, abdomen, limbs, and the like of the human body are imaged in a one-dimensional or in three-dimensional manner by imaging the information regarding the spatial distribution of the proton density or the spatial distribution of the relaxation time of the excitation state.

The MRI apparatus of the present embodiment performs processing for the polarity inversion of the slice selection gradient magnetic field, processing for the setting of the irradiation frequency of the RF pulse, and calculation of an excitation region by the RF pulse or the presaturation pulse (hereinafter, referred to as a Presat pulse), which will be described later. In order to realize this, the control information processing system 70 can perform processes related to various functions, and can control the sequencer 10, the display 71, the optical disk 72, the magnetic disk 73, the ROM 74, and the RAM 75. Although not shown in FIG. 1, it is possible to perform the exchange of information with other devices when necessary.

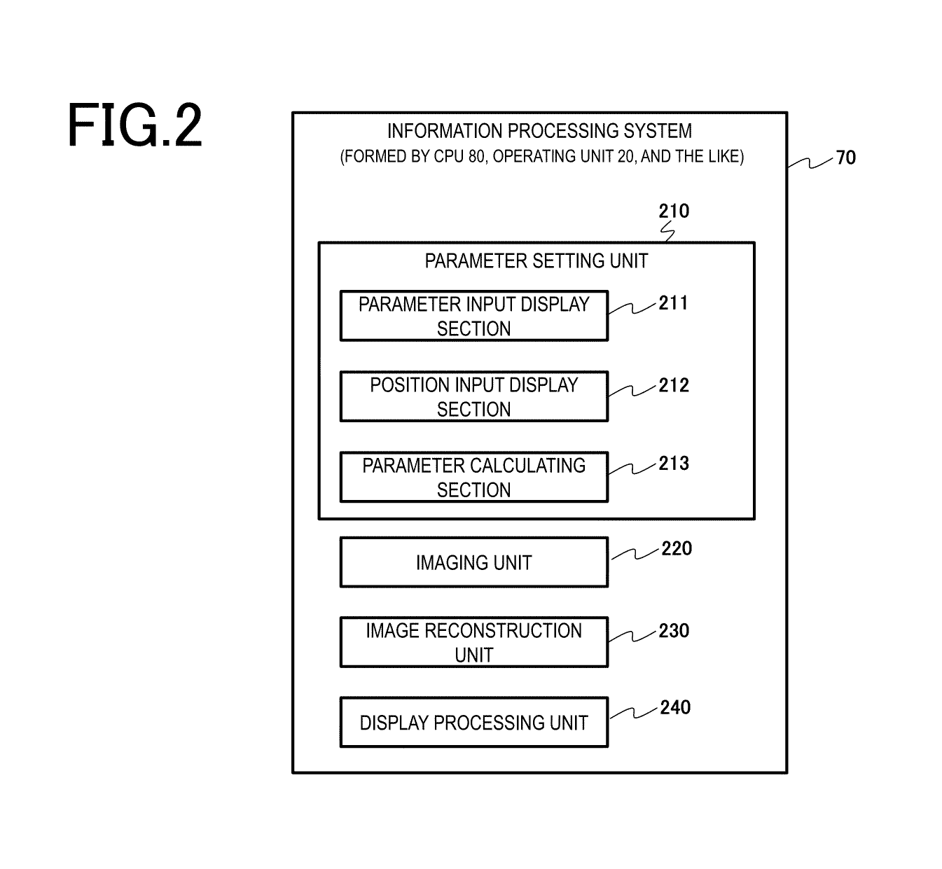

FIG. 2 is a functional block diagram showing the main function of the control information processing system 70. The control information processing system 70 includes a parameter setting unit 210, an imaging unit 220, an image reconstruction unit 230, and a display processing unit 240. In addition, the parameter setting unit 210 includes a parameter input display section 211 that receives an input of values or selected items and performs required display in response to the reception, a position input display section 212 that receives an input regarding a position, such as region designation, and performs required display in response to the reception, and a parameter calculating section 213 that calculates parameters used in imaging based on the information set by the input from the parameter input display section 211 or the position input display section 212. Processing performed by the parameter calculating section 213 will be described below. “Calculation” in this specification includes not only processing based on calculation but also processing for acquiring the required data from known data, which is stored in advance, by the search.

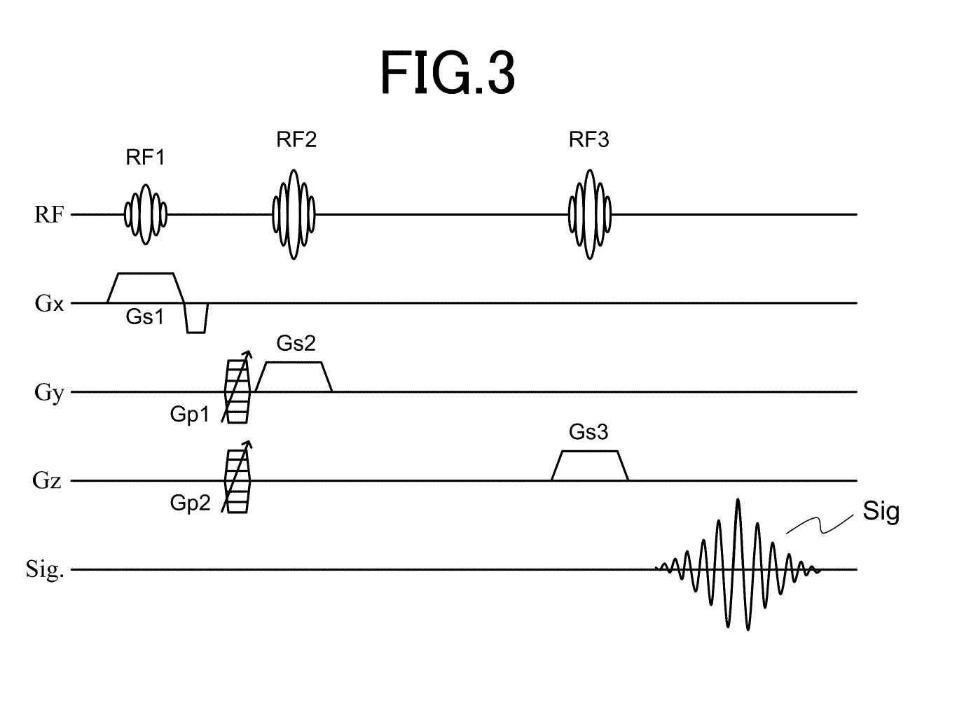

FIG. 3 is a diagram showing an example of a typical pulse sequence used in the MRSI measurement.

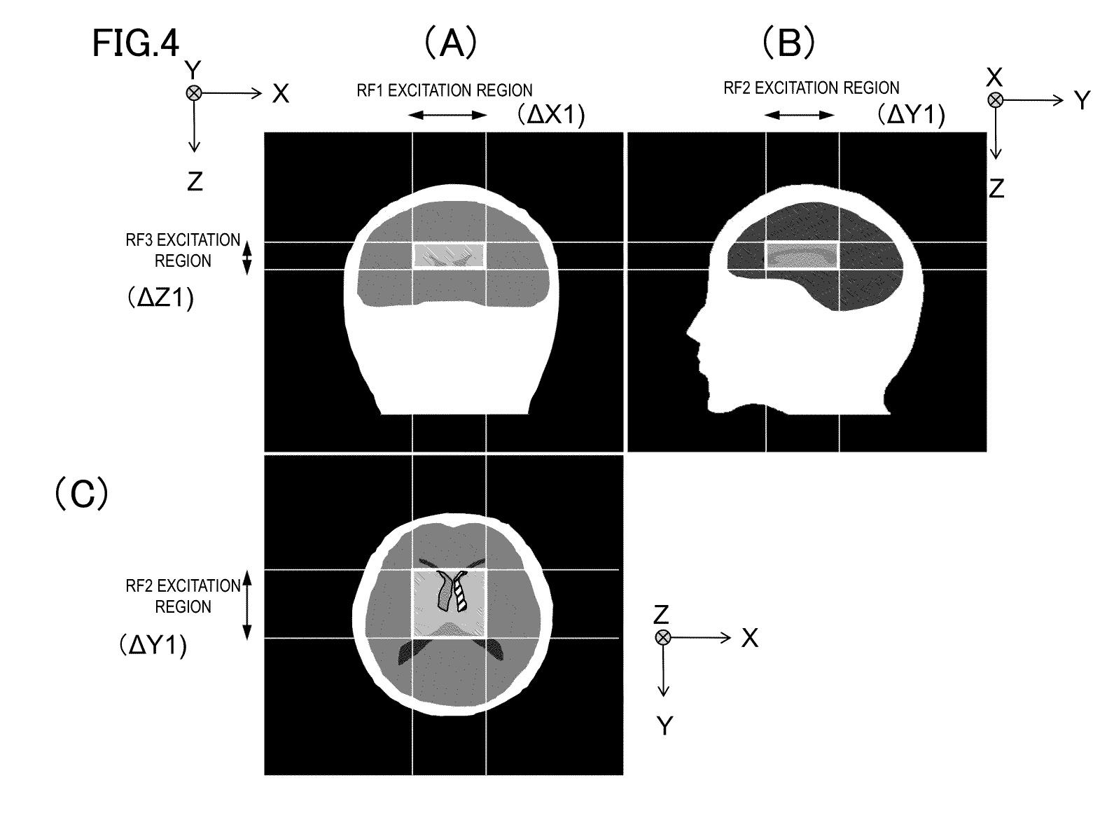

As shown in FIG. 3, three RF pulses (RF1, RF2, RF3) are applied to the object 1 and slice selection gradient magnetic fields (Gs1, Gs2, Gs3) are applied to the object 1 in three directions of X, Y, and Z axes with respect to the three RF pulses (RF1, RF2, RF3). Accordingly, three slice planes perpendicular to each other are excited as shown in FIG. 4, and the echo signal (Sig) based on the NMR phenomenon is acquired from the three-dimensional volume of interest.

FIG. 4 is a diagram for explaining a volume of interest set by the excitation of three slice planes perpendicular to each other with the head of a person as an example. The Z axis is an axis of the object 1 in the body axis direction, the X axis is a horizontal axis, and the Y axis is a vertical axis. In FIG. 4, for example, an image of the head is displayed on the display 71, and a volume of interest can be set by inputting an excitation region of three slice planes perpendicular to each other based on the image displayed on the display 71 with the pointing device 21 or the like. Although the head of a person is shown as an example in FIG. 4, it is also possible to use other examination part.

FIG. 4(A) is a diagram showing a volume of interest of the head on the X-Z plane, and a volume of interest on the X-Z plane is set by designating an RF1 excitation region (ΔX1) in the X-axis direction and designating an RF3 excitation region (ΔZ1) in the Z-axis direction. FIG. 4(B) is a diagram showing a volume of interest of the head on the Z-Y plane, and a volume of interest on the Z-Y plane is similarly set by designating the RF3 excitation region (ΔZ1) in the Z-axis direction and designating an RF2 excitation region (ΔY1) in the Y-axis direction. FIG. 4(C) is a diagram showing a volume of interest of the head on the Y-X plane, and a volume of interest on the Y-X plane is similarly set by designating the RF2 excitation region (ΔY1) in the Y-axis direction and designating the RF1 excitation region (ΔX1) in the X-axis direction. As described above, a volume of interest (VOI) of the head is set.

FIG. 3 will be described in detail. First, the gradient magnetic field Gx will be applied in the X-axis direction simultaneously with the application of the RF1 pulse. The thickness of the slice in the X-axis direction, that is, the RF1 excitation region, depends on the frequency band of the RF1 pulse and the application strength of the gradient magnetic field Gx. After setting the slice plane in the X-axis direction, phase gradient magnetic field pulses (Gp1, Gp2) are applied in order to obtain the position information in the Y-axis direction and the Z-axis direction. Then, the thickness of the slice in the Y-axis direction (RF2 excitation region) is set by applying the gradient magnetic field Gy in the Y-axis direction simultaneously with the application of the RF2 pulse, and then the thickness of the slice in the Z-axis direction (RF3 excitation region) is set by applying the gradient magnetic field Gz in the Z-axis direction simultaneously with the application of the RF3 pulse. By the application of the RF pulse and the gradient magnetic field, the echo signal (Sig) is obtained. Phase encoding is applied by the number of phase direction matrices while increasing one step at a time whenever a signal is received. By processing the obtained echo signal (Sig) by repeating the above-described step, an MRI image is obtained.

As described above, as an irradiation frequency used in the irradiation of these RF pulses, a frequency in the range of the resonance frequency of water or a frequency in the range of the resonance frequency of the metabolite to be measured, for example, the resonance frequencies of inositol, choline, creatine, glutamine, glutamic acid, GABA, NAA, and lactic acid in the case of the head, is used. If the irradiation frequency and the resonance frequency of the metabolite are different, resonance occurs at a frequency shifted from the irradiation frequency. As a result, an MR signal is generated from a position shifted from the region set on the positioning image.

An excitation position shift between metabolites A and B will be described below when the irradiation frequency ω of the used RF1 is set between the resonance frequency of the metabolite A having the lowest resonance frequency among metabolites to be measured and the resonance frequency of the metabolite B having the highest resonance frequency, a difference from ω to the resonance frequency of the metabolite A is Δω_{A}, and a difference from ω to the resonance frequency of the metabolite B is Δω_{B}, with the excitation slice of RF1 in the MRS measurement or MRSI measurement of the head of a person as an example.

The frequency ω at a position X when the linear gradient magnetic field Gx is applied in the X direction of the space is expressed as the following equation.

ω=γGx·X [Equation 1]

Here, assuming that the excitation position shifts of the metabolites A and B are ΔX_{A} and ΔX_{B}, ΔX_{A} and ΔX_{B} can be expressed as the following equation using Δω_{A} and Δω_{B}.

ω−Δω_{A}=γGx(X−ΔX_{A})

ω+Δω_{B}=γGx(X+ΔX_{B}) [Equation 2]

From the above, the excitation position shifts ΔX_{A} and ΔX_{B} of the metabolites A and B can be expressed as the following equation from (Equation 1) and (Equation 2).

ΔX_{A}=Δω_{A}/γGx

ΔX_{B}=Δω_{B}/γGx [Equation 3]

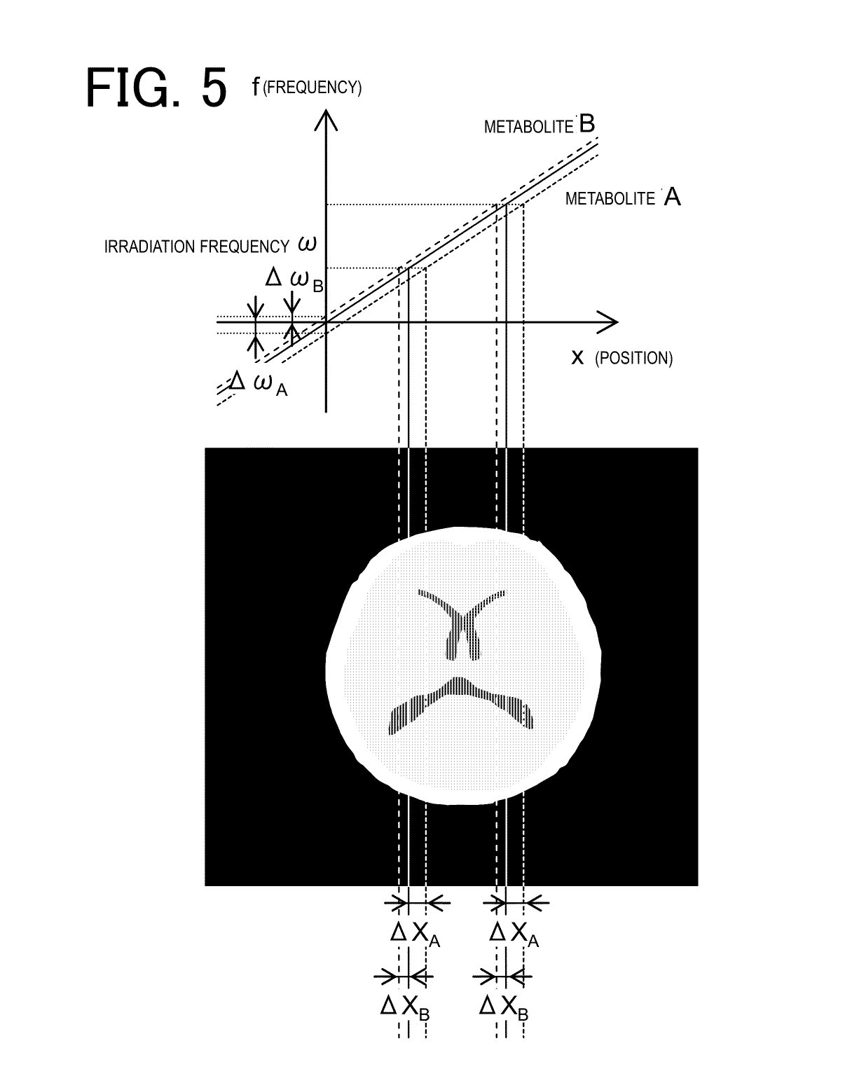

FIG. 5 is a diagram that shows the relationship (Equation 1) between the frequency of the RF1 and the position and shows the excitation position shift (Equation 3). For example, when the user sets a volume of interest (VOI) on the user interface based on the image described in FIG. 4, the set region, that is, a region set by the irradiation frequency of the RF1 is shown by the solid line and the excitation frequencies and excitation regions of the metabolites A and B are shown by the broken line. A region ΔX1 set by the user corresponds to an RF1 excitation region (ΔX1) in FIG. 4(A). As can be seen from the diagrams, the application strength (Gx) indicates the slope of the solid line, and the slope increases and the excitation region of the RF1 decreases as the application strength Gx increases.

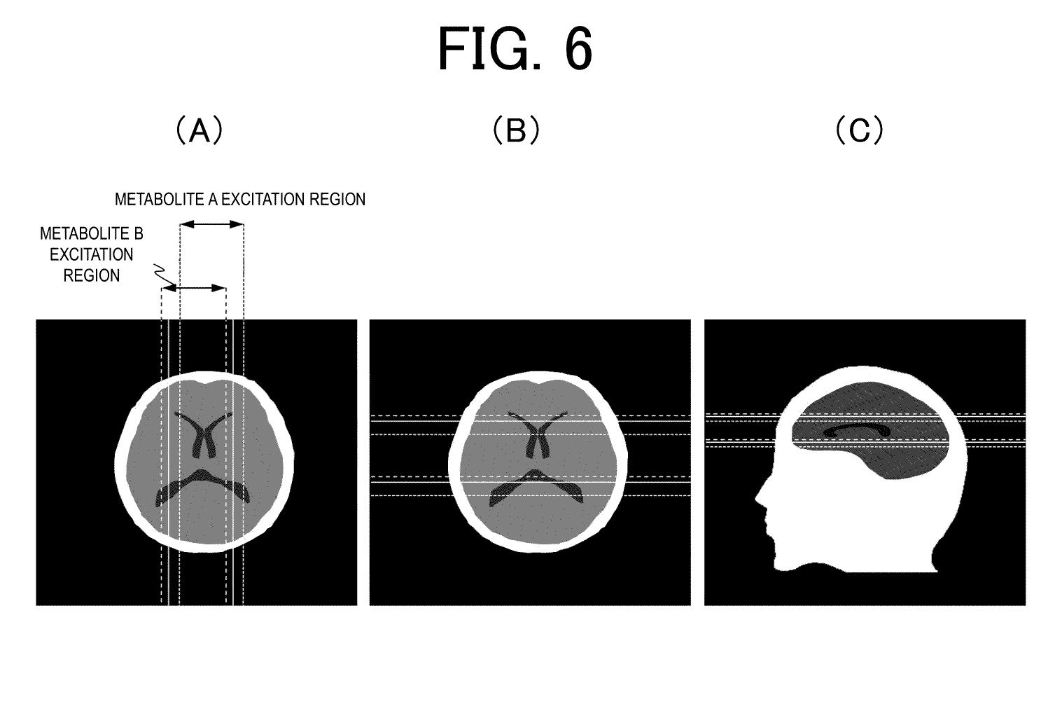

FIG. 6 shows an excitation position shift in each cross-section by the three RF pulses (RF1, RF2, RF3). FIG. 6(A) shows an excitation position shift in the RF1 described in FIG. 5. The RF1 excitation region set by the user is ΔX1 interposed between the solid lines. As can be seen from FIG. 6(A), the excitation region of the metabolite A is shifted to the right (to the larger gradient magnetic field Gx) from the set RF1 excitation region, and the excitation region of the metabolite B is shifted to the left (to the smaller gradient magnetic field Gx) from the set RF1 excitation region.

FIGS. 6(B) and 6(C) show an excitation position shift in the RF2 and RF3 using the same expression method as 6(A). Similar to FIG. 6(A), the RF2 excitation region or the RF3 excitation region set by the user is expressed as a region interposed between the solid lines, and the excitation region of the metabolite A is expressed as a region interposed between the dotted lines and the excitation region of the metabolite B is expressed as a region interposed between the broken lines. Similar to FIGS. 6(B) and 6(C), it can also be seen that the excitation regions of the metabolites A and B are shifted from the set excitation regions.

In the conventional measurement method, in MRS measurement or MRSI measurement, in a single measurement, for a volume of interest set on the user interface by the user, the excitation of the region and the acquisition of the echo signal (Sig) are repeatedly performed, for example, over hundreds of times in the pulse sequence shown in FIG. 3. In this case, as shown in FIGS. 5 and 6, for each metabolite, excitation occurs in a region that is always shifted in a fixed direction. Therefore, in the conventional measurement method, the measurement result of a region that is always shifted in one direction with respect to the volume of interest to be originally measured is repeatedly obtained as a measurement result of the volume of interest to be measured.

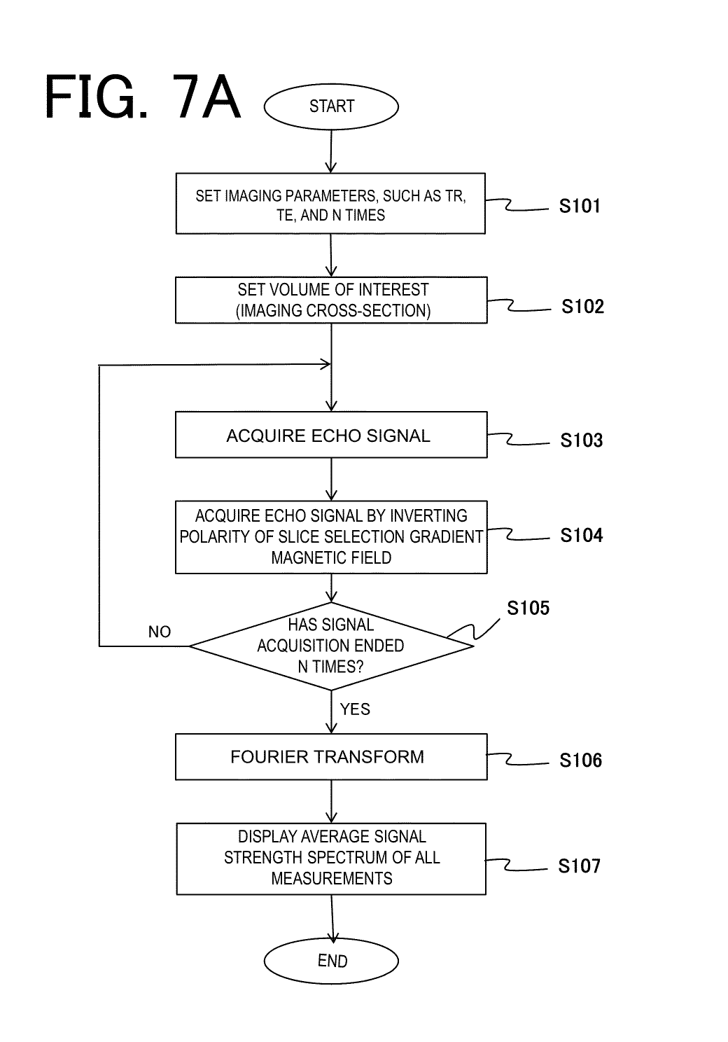

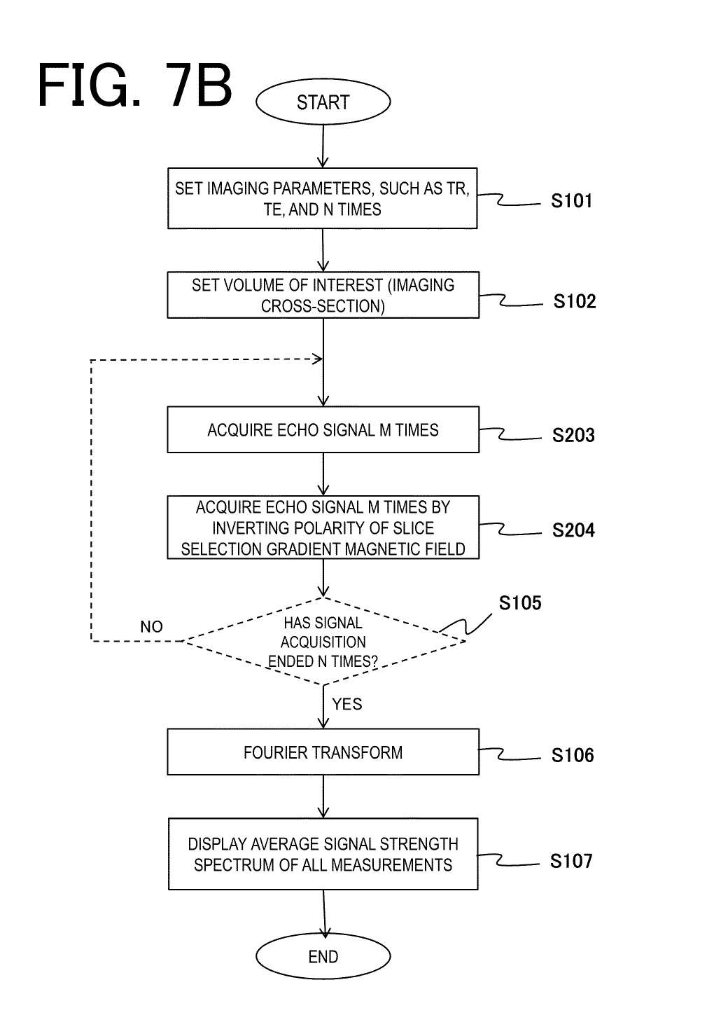

FIGS. 7A and 7B are flowcharts showing the process for solving the aforementioned problem. First, the operation procedure of FIG. 7A will be described. In step S101, a repetition time TR, an echo time TE, and the number of repetitions N are set through the input operation of the operator using the operating unit 20. The number of repetitions N is the number of times of receiving the spin signal, and is set in units of a few hundred, such as 100 to 200 times.

Then, in step S102, as described in FIG. 4, the volume of interest (VOI) of the imaging cross-section is set through the input operation of the operator using the operating unit 20. As described above, an excitation region due to the irradiation of the RF1 pulse, an excitation region due to the irradiation of the RF2 pulse, and an excitation region due to the irradiation of the RF3 pulse are set by the operator. After the volume of interest is set, the irradiation frequencies of the RF1 to RF3 pulses and the strengths of the slice selection gradient magnetic fields Gs1 to Gs3 are determined.

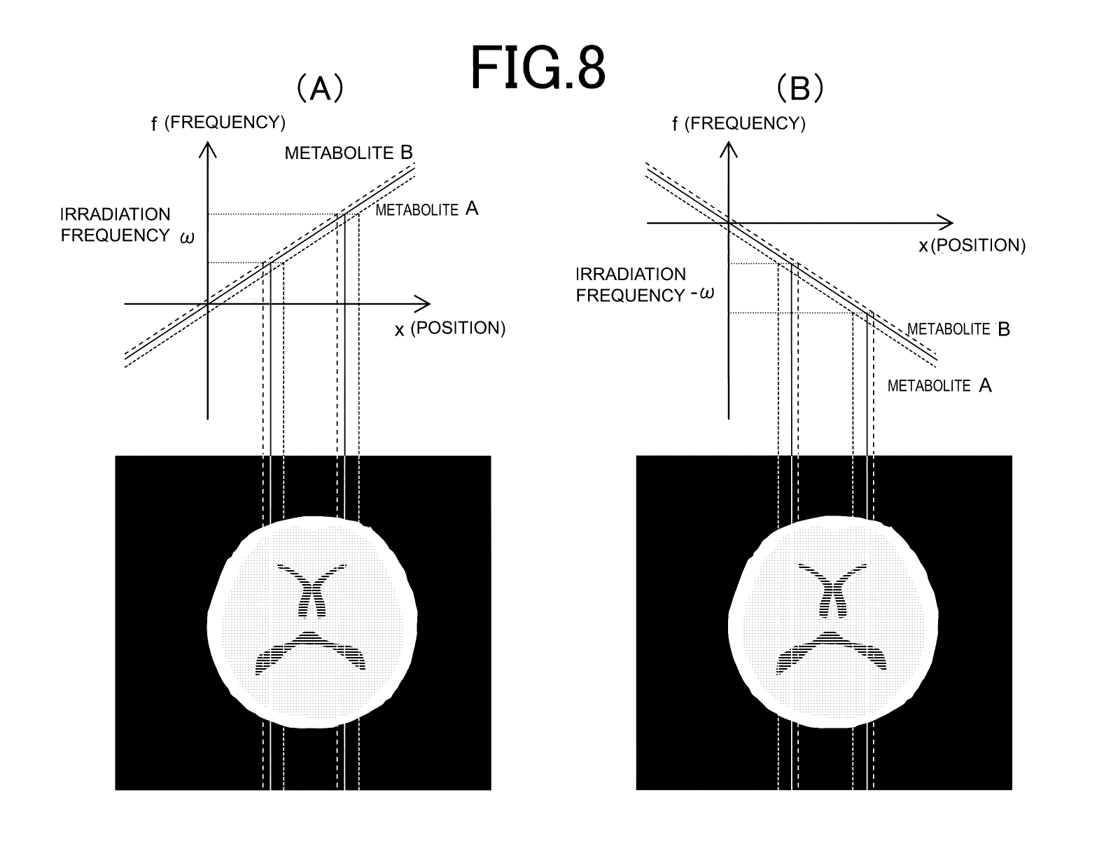

In step S103, the echo signal (Sig) is acquired once according to the pulse sequence described in FIG. 3. Then, in step S104, the polarities of the slice selection gradient magnetic fields Gs1 to Gs3 are inverted. For example, for the region excited by RF1, the excitation and the acquisition of the echo signal (Sig) that are repeatedly performed are performed while repeating measurement 1 shown in FIG. 8(A) and measurement 2 shown in FIG. 8(B).

Although several combinations, that is, combinations of up to eight types are generated if the polarity of each slice selection gradient magnetic field is inverted for the slice selection gradient magnetic fields Gs1 to Gs3, a case will be representatively described in which the polarity of one slice selection gradient magnetic field, for example, the polarity of the slice selection gradient magnetic field Gs1 is inverted. The slice selection gradient magnetic field Gs2 or the slice selection gradient magnetic field Gs3 can be similarly considered, even though the axis is different from that when the polarity of the slice selection gradient magnetic field Gs1 is inverted.

First, the echo signal (Sig) is detected by performing measurement in one polarity state of the slice selection gradient magnetic field Gs1 (hereinafter, referred to as measurement 1). In the measurement 1, a region that is actually excited to generate the echo signal (Sig) is shifted in one direction from the volume of interest (VOI) as described in FIG. 5 or FIG. 8(A). Then, the echo signal (Sig) is detected by performing measurement in a state in which the polarity of the slice selection gradient magnetic field Gs1 is inverted (hereinafter, referred to as measurement 2). Although the details of FIG. 8(B) will be described later, since the polarity of the slice selection gradient magnetic field Gs1 is inverted, the region that is actually excited to generate the echo signal (Sig) is shifted in the other direction opposite to the one direction from the volume of interest (VOI). Thus, the region that is actually measured for the volume of interest (VOI) is not shifted in only one direction, but the measurement 2 is performed with the same number of times as in the measurement 1. Therefore, since the measured region is shifted in opposite directions at the same rate for the volume of interest (VOI), it is possible to reduce the influence of contamination signals from the outside of the volume of interest.

FIG. 8(B) is a diagram for explaining the relationship between the volume of interest (VOI) and the actually measured region in the measurement 2. In the measurement 2 shown in FIG. 8(B), the polarity of the slice selection gradient magnetic field Gs1 is inverted from that in the measurement 1. In this case, the frequency at the position X is −ω from (Equation 1). In this case, since the magnitude relationship between −ω and the metabolites A and B is not changed from that in the measurement 1, the position shift direction of each metabolite is inverted to cause excitation in the measurement 2.

By performing the same control for RF2 and RF3 shown in FIGS. 6(B) and 6(C) to control the position shift direction in the three directions of X, Y, and Z, it is possible to acquire the echo signal (Sig) from the excitation positions of up to eight patterns as a three-dimensional volume. When the shift direction is inverted, signals outside the volume of interest on the opposite side are mixed. However, it is rare that unnecessary metabolites are present in all directions. As a result, it is possible to reduce contamination signals from the outside of the volume of interest that is generated from a specific direction. The control of inverting the position shift direction may be performed in the excitation slices of all of three cross-sections, or may be performed for limited cross-sections of one or two cross-sections instead of being performed in the excitation slices of all of the three cross-sections. A large effect is obtained by performing the control of inverting the position shift direction in the excitation slices of all of the three cross-sections. However, even if the control of inverting the position shift direction is performed for limited cross-sections of one or two cross-sections as described above, a sufficient effect is obtained in many cases since it is rare that unnecessary metabolites are present in all directions.

As a method of inverting the polarities of the slice selection gradient magnetic fields Gs1 to Gs3, there are eight patterns involving a pattern in which no polarity is inverted. For example, there is a pattern in which “the slice selection gradient magnetic field Gs1 is inverted, the slice selection Gs2 is not inverted, and the slice selection Gs3 is inverted”. In step S104, signals are acquired by inverting the polarities of the slice selection gradient magnetic fields Gs1 to Gs3 in all of these inversion patterns (seven patterns).

In FIG. 7A, in step S105, it is determined whether or not steps S103 and S104 described above have been performed repeatedly N times. When steps S103 and S104 have not been performed N times, steps S103 and S104 described above are repeatedly performed. In this case, phase encoding is applied by the number of phase direction matrices (N) while increasing one step at a time as described above.

In the MRS measurement in which phase encoding is not performed, data may be classified into measurement patterns after acquiring all echo signals (Sig), and real components of the one-dimensional spectrum obtained by performing zero filling, one-dimensional inverse FFT, and phase correction with respect to the time-series data in which addition processing is performed may be presented to the user so that the user performs a selection among the real components. In step S106, the obtained signals are subjected to a Fourier transform. In step S107, a signal strength spectrum is displayed by averaging all the measurement results that have been obtained. The horizontal axis indicates a position, and the vertical axis indicates a signal strength. The method in which the obtained signals are subjected to a Fourier transform in step S106 and the results obtained by the Fourier transform are averaged in step S107 is an example, and other calculation processes may be performed as a method of reducing the influence of contamination signals from the outside of the volume of interest.

In step S107 described in detail, an example of displaying the graph of a spectrum based on the obtained measurement results is shown. However, this is an example, and an image showing the distribution state of substances such as metabolites, may be displayed. In addition, it is possible to obtain various images or graphs by performing various kinds of processing based on the measurement results. Through the present embodiment or embodiments described below, it is possible to measure the more accurate distribution state of substances such as metabolites. By processing further various kinds of processing using the measurement results, it is possible to obtain the more accurate information that is useful for diagnosis.

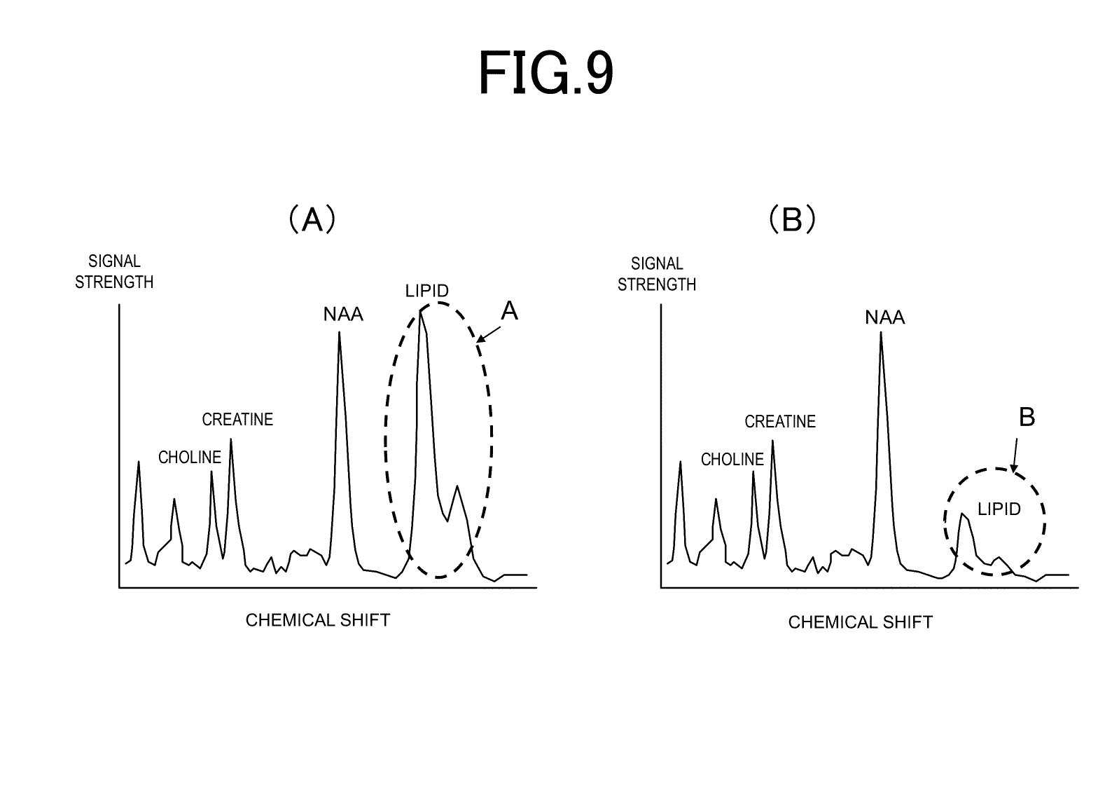

FIG. 9 is a graph showing an example of the measurement result of the MRS measurement in the head of a person. FIG. 9(A) is a case in which only the measurement 1 described in FIG. 8(A) is performed and the measurement 2 described in FIG. 8(B) is not performed. A state in which the position of an excitation region is shifted due to chemical shift and a lipid signal outside the volume of interest is mixed is displayed as indicated by the broken line A in the diagram. On the other hand, FIG. 9(B) is a measurement result obtained by repeating the measurement 1 described in FIG. 8(A) and the measurement 2 described in FIG. 8(B) and taking the average, as described above in the flowchart shown in FIG. 7A. In the measurement result shown in FIG. 9(B), the influence of the mixing of the lipid signal outside the volume of interest shown by the broken line A in FIG. 9(A) is sufficiently reduced, as indicated by the broken line B in FIG. 9(B).

As described above, for example, in the method of the flowchart shown in FIG. 7A, the influence of contamination signal from the outside of the volume of interest can be reduced by performing both of the measurement 1 and the measurement 2 described in FIG. 8(A) or FIG. 8(B) and acquiring the target measurement result from the measurement results of both of the measurement 1 and the measurement 2.

In the flowchart shown in FIG. 7A, the acquisition of the echo signal (Sig) by the measurement 1 described in FIG. 8(A) and the acquisition of the echo signal (Sig) by the measurement 2 described in FIG. 8(B), in which the polarity of the gradient magnetic field is inverted, are alternately performed. However, this is an example. Even if the measurement 1 and the measurement 2 are not alternately performed, the effect of the present invention can be obtained if the measurement results of the measurement 1 and the measurement 2 can be reflected. For example, also in the method shown in the flowchart of FIG. 7B described below, the same effect can be obtained. In FIG. 7B, the same reference numerals as in FIG. 7A indicate the same operation procedures. In FIG. 7B, step S105 is shown by the broken line. Using FIG. 7B, both of a method in which step S105 is used and a method in which step S105 is not used will be described.

In the flowchart of FIG. 7B, the number of times to acquire the echo signal (Sig) eventually is assumed to be N times as in FIG. 7A. First, in the method in which step S105 shown by the broken line is not used, the number of times M to acquire the echo signal (Sig) in step S203 is set to the value of the half of N times described above. Similarly, the number of times M to acquire the echo signal (Sig) in step S204 is set to the value of the half of N times described above. The operations of steps S101 and S102 are the same as those in FIG. 7A.

Then, in step S203, the echo signal (Sig) is acquired M times, which is the half of N times, with the polarity of the slice selection gradient magnetic field shown in the measurement 1 described in FIG. 8(A). In step S204, the echo signal (Sig) is acquired M times, which is the half of the remaining N times, by the measurement 2 in a state of the polarity of the slice selection gradient magnetic field described in FIG. 8(B). Then, the echo signal (Sig) by the measurement 1 and the measurement 2 acquired in step S203 or step S204 is calculated in step S106 and is calculated in step S107, and the display of the strength of the signal with respect to the spectrum is performed as shown in FIG. 9(B). In addition, the graphical display of the spectral strength in step S107 is an example, and various kinds of displays to display the state of substances, such as metabolites, as described above are possible.

Next, a method will be described in which the echo signal (Sig) by the measurement 1 or the measurement 2 is acquired by the predetermined number of times less than the required number of times instead of assuming the number of times to acquire the echo signal (Sig) eventually to be N times as in FIG. 7A in the flowchart of FIG. 7B and acquiring the echo signal (Sig) by the measurement 1 or the measurement 2 by the half of the required number of times continuously at a time. In this method, step S105 shown by the broken line is used.

The operations of steps S101 and S102 are the same as those in FIG. 7A. In step S203 or step S204, M times that is the number of times to acquire the echo signal (Sig) is set to the smaller value instead of the value of the half of N times described above. Here, the number of times M is a common divisor for the value of the half of N times described above. Thus, the number of times M to acquire the echo signal (Sig) in step S203 or step S204 is set.

In step S203, the echo signal (Sig) is acquired M times continuously with the polarity of the slice selection gradient magnetic field shown in the measurement 1 described above. Then, in step S204, the echo signal (Sig) is similarly acquired M times continuously in a state of the measurement 2 in which the polarity of the slice selection gradient magnetic field described in FIG. 8(B) is inverted.

Then, in step S105 shown by the broken line, it is determined whether or not the number of times of acquisition of the echo signal (Sig) has reached N times, that is, whether or not the acquisition of the required echo signal (Sig) has been completed. When the acquisition of the required echo signal (Sig) has not been completed, step S203 or step S204 is performed again. Step S105 shown by the broken line is the same function as step S105 shown in FIG. 7A.

When it is determined that the acquisition of the required echo signal (Sig) has been completed in step S105 shown by the broken line, step S106 or step S107 is performed and the signal strength of the spectrum described in FIG. 9(B) is displayed. Although the operation of the Fourier transform in step S106 is performed after the acquisition of the required echo signal (Sig) in FIG. 7A or FIG. 7B, this is an example. The operation of the Fourier transform may be sequentially performed while acquiring the echo signal (Sig). As described above, the example of calculating and displaying the signal strength of the spectrum in step S107 is an example. If the distribution state of substances, such as metabolites, can be measured more accurately by applying the present embodiment and the following embodiments, it is possible to obtain information by performing various kinds of processing based on the measurement result and to display the information.

### Second Embodiment

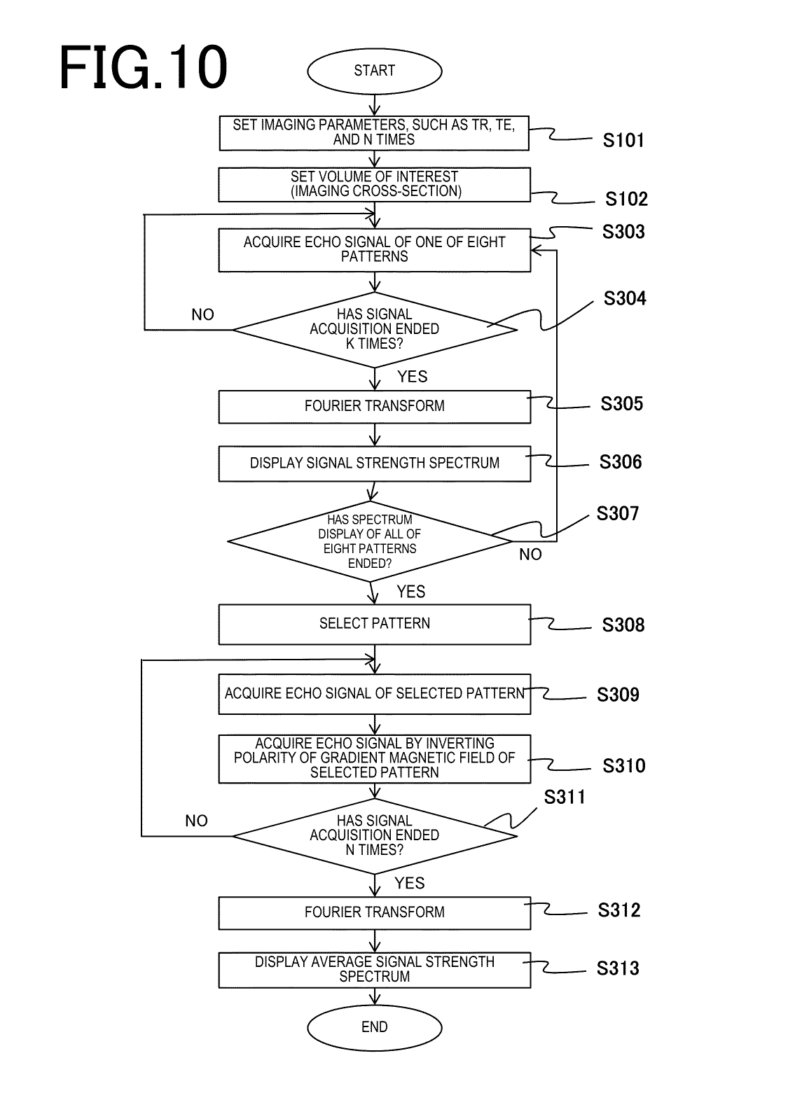

Next, a second embodiment will be described with reference to the flowchart of FIG. 10. Only different steps from the flowchart in the first embodiment will be described below. In the first embodiment, if all patterns involving a pattern in which no polarity is inverted are performed as a method of inverting the polarities of the slice selection gradient magnetic fields Gs1 to Gs3, the inversion of the gradient magnetic field of eight patterns is performed. In the second embodiment, it is selected first which measurement pattern among the eight patterns is effective for calculation. One of the eight patterns is arbitrarily selected in step S303, and measurement is performed K times in step S304. Then, a Fourier transform is performed in step S305, and the real component of the one-dimensional spectrum is displayed in step S306. The processing of the Fourier transform includes zero filling, one-dimensional inverse FFT, phase correction, and the like. Here, step S303 is pre-measurement for selecting a pattern to be performed in the following step S308, and it is possible to obtain a sufficient result with the small number of measurements K. By setting the number of pre-measurements K to about once to 10 times, a possibility that the information enabling the selection of a pattern to be performed in step S308 will be obtained is increased.

In step S307, it is determined whether or not the spectrum display in all of the eight patterns described above has been performed. When the spectrum display in all of the eight patterns described above has not been performed, the process of steps S303 to S306 is repeated. After performing the spectrum display for the pattern of the type set in advance or for all of the eight patterns, the user selects an appropriate spectrum or a plurality of acceptable spectra among the spectra of the displayed measurement patterns in step S308.

Steps S101 to S308 described above are steps of pre-measurement before proceeding to the main measurement. In the main measurement, the same process as the process performed in the first embodiment is performed. This will be described through the following steps S309 to S313.

In step S309, the echo signal (Sig) is acquired with the selected measurement pattern. In step S310, the echo signal (Sig) is acquired by inverting the polarity of the slice selection gradient magnetic field according to the selected measurement pattern. In step S311, it is determined whether or not the number of times of signal acquisition in steps S309 and S310 has reached N times, that is, whether or not the acquisition of all echo signals (Sig) set in step S101 has been completed. When the acquisition of all echo signals (Sig) set in step S101 has not been completed, steps S309 and S310 are repeated to acquire the echo signal (Sig) repeatedly. When the acquisition of all echo signals (Sig) set in advance has been completed, the process proceeds to step S312 from step S311. The acquired echo signal (Sig) is subjected to the Fourier transform in step S312, and the signal strength of the spectrum is displayed from all of the acquired measurement results in step S313. The processing method of averaging the echo signal (Sig) acquired with one gradient magnetic field polarity described as the measurement 1 and the echo signal (Sig) acquired with the inverted gradient magnetic field polarity described as the measurement 2 is one method. However, processing using other methods may also be performed without being limited to the processing method for averaging. In addition, as described above, it is possible to perform more accurate measurement through the present embodiment. Therefore, it is possible to further perform various kinds of processing using the measurement result, and the display of the signal strength of the spectrum is an example of the various kinds of processing.

Here, since the process of steps S309 to S311 has already been performed in the pre-measurement (S303 and S304), these steps may be omitted and the data of pre-measurement may be used.

As described above, it is possible to reduce the influence of contamination signals from the outside of the volume of interest.

### Third Embodiment

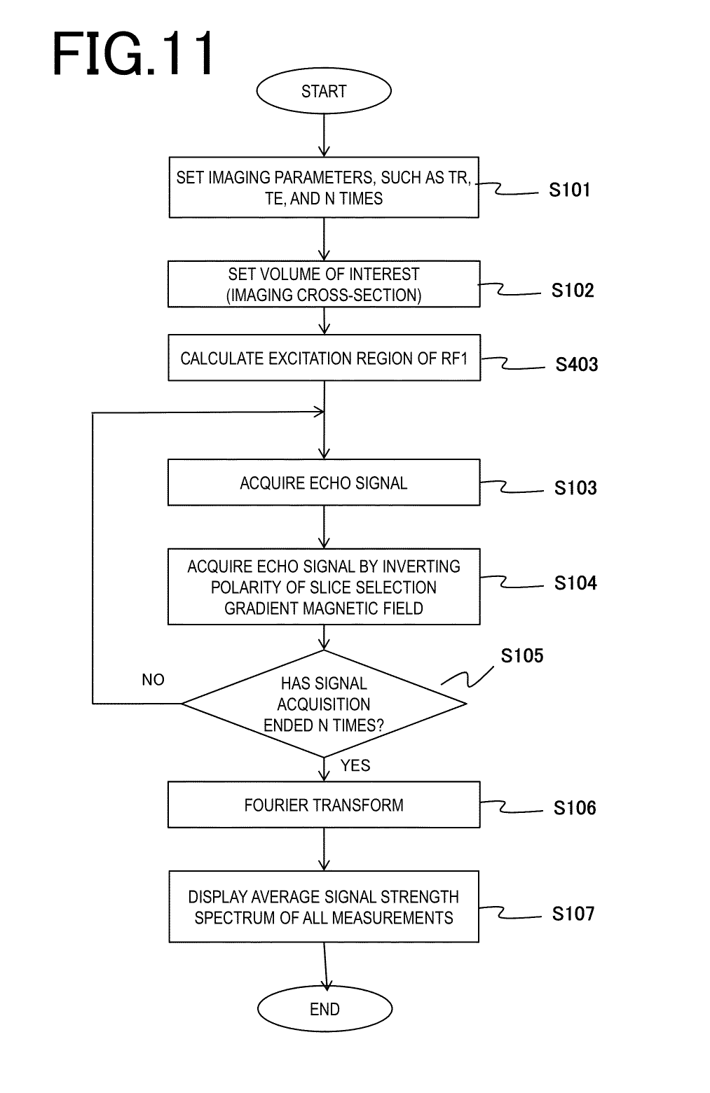

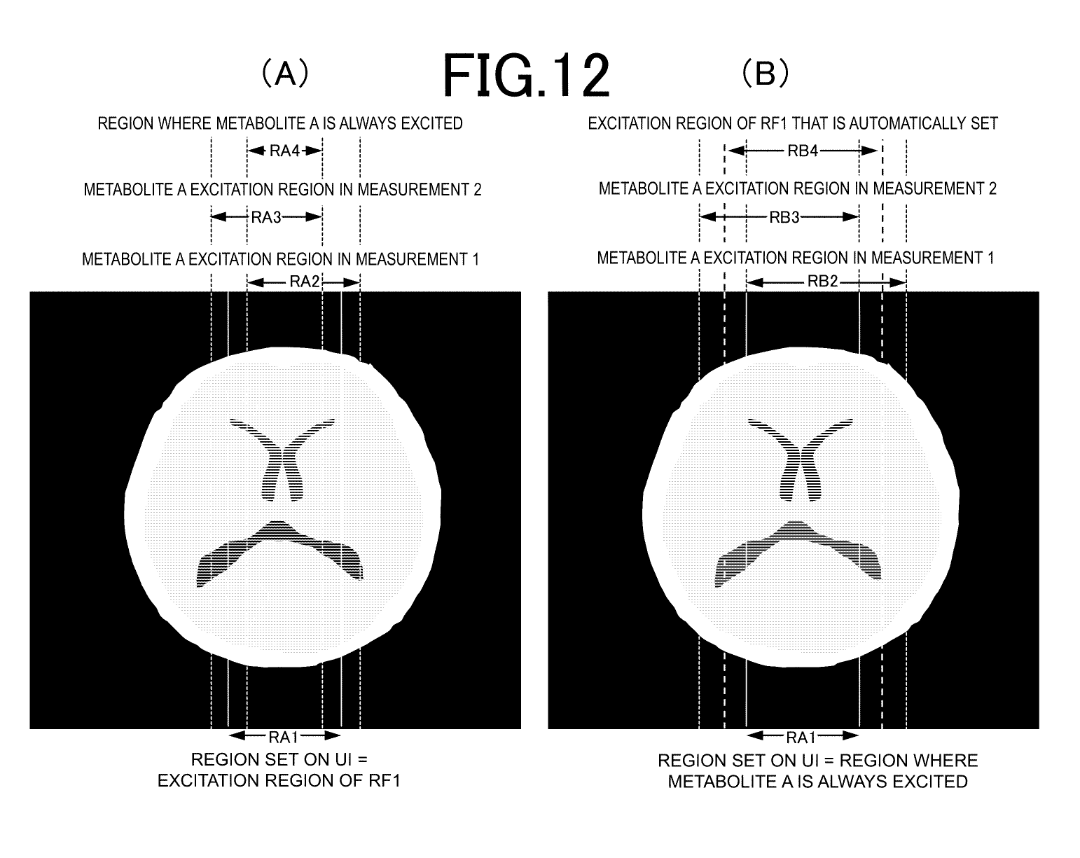

Next, a third embodiment will be described with reference to FIGS. 11 and 12. The third embodiment is the same as the first embodiment up to the setting of a volume of interest in step S102, but is different from the first embodiment in that, for example, the excitation region of RF1 is automatically calculated and set in step S403 thereafter. Subsequent steps from step S103 of acquiring the echo signal (Sig) to step S107 of calculating and displaying the signal strength of a spectrum are the same as those in the first embodiment. Next, details of step S403 and the reason why step S403 is provided will briefly be described. Although the excitation region that is automatically calculated and set is a region excited by RF1, this is just a representative example, and the third embodiment can also be similarly applied to regions excited by RF2 or RF3. In addition, the setting based on the automatic calculation may be applied to all excitation regions of RF1, RF2, and RF3, or the setting based on the automatic calculation may be applied to any two regions of the excitation regions.

It is desirable to be able to specify a metabolite, which has a resonance frequency farthest from the irradiation frequency, among metabolites to be measured and to more accurately measure the signal strength including the metabolite. Here, the metabolite having a resonance frequency farthest from the irradiation frequency among the metabolites to be measured is assumed to be the metabolite A. The metabolite A having a resonance frequency farthest from the irradiation frequency is a metabolite having the largest excitation region shift amount.

FIG. 12(A) shows an excitation region excited by RF1. The shift of an excitation region for the metabolite A when performing measurement using the methods of the measurement 1 and the measurement 2 described in FIG. 8(A) or FIG. 8(B) will be described. The excitation region of RF1 set through the user interface by the operator is RA1. In the measurement 1 in which the resonance frequency of the metabolite A is shifted and the polarity of the slice selection gradient magnetic field shown in FIG. 8(A) is one polarity, the excitation region of the metabolite A is shifted to become RA2. Then, as shown in FIG. 8(B), the echo signal (Sig) is detected by inverting the polarity of the slice selection gradient magnetic field. In this case, the excitation region is shifted to become RA3.

When the signal strength of the spectrum is calculated using the method described in the first embodiment, a region RA4 where the region excited in the measurement 1 and the region excited in the measurement 2 overlap each other is always excited. However, since the other excitation regions are not excited for the half of the number of times of detection of the echo signal (Sig), a sufficient amount of echo signals (Sig) are not obtained from the other excitation regions. Accordingly, in practice, the detection of the metabolite A is performed in the region RA4 narrower than the excitation region RA1 of RF1 set through the user interface by the operator.

Therefore, the excitation region of RF1 is calculated in consideration of the shift of the resonance frequency of the metabolite A, in such a manner that the metabolite A is always excited in the region RA1 set on the user interface.

The relationship between the resonance frequency shift in the measurement 1 or the measurement 2 and the shift of the excitation region can be calculated from the relationship shown in FIG. 8(A) or FIG. 8(B). Therefore, an excitation region RB4 of RF1 for the metabolite A to be always excited is determined by calculation. When the excitation region RB4 of RF1 for the metabolite A to be always excited is set, the region where the metabolite A is excited in the measurement 1 becomes an excitation region RB2 of FIG. 12(B). In addition, the region where the metabolite A is excited in the measurement 2 becomes an excitation region RB3 of FIG. 12(B). Accordingly, the metabolite A is always excited in the region RA1 set on the user interface.

Therefore, even if the user does not know the amount of shift of the excitation position of each metabolite, signals of all metabolites included in the volume of interest can always be measured without shortage. However, the excitation region RB4 of RF1 set by the calculation becomes wider than the volume of interest RA1 set on the user interface by the user. For this reason, extra signals are also measured. This problem can be solved by applying a fourth embodiment shown below.

In the present embodiment, the excitation region of RF1 has been described as a representative example. However, the third embodiment can also be similarly applied to the excitation regions of RF2 and RF3. In the same manner as described in the other embodiments, the third embodiment can be applied to all excitation regions of RF1 to RF3, and can be selectively applied to a plurality of regions of the excitation regions of RF1 to RF3.

### Fourth Embodiment

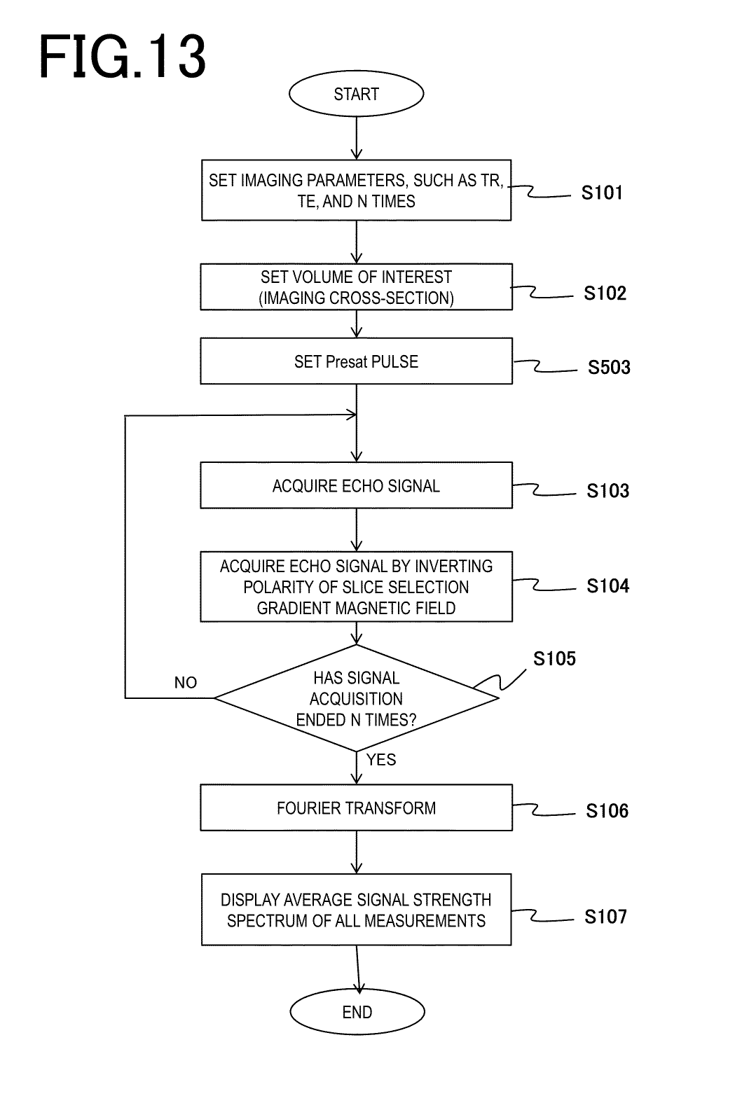

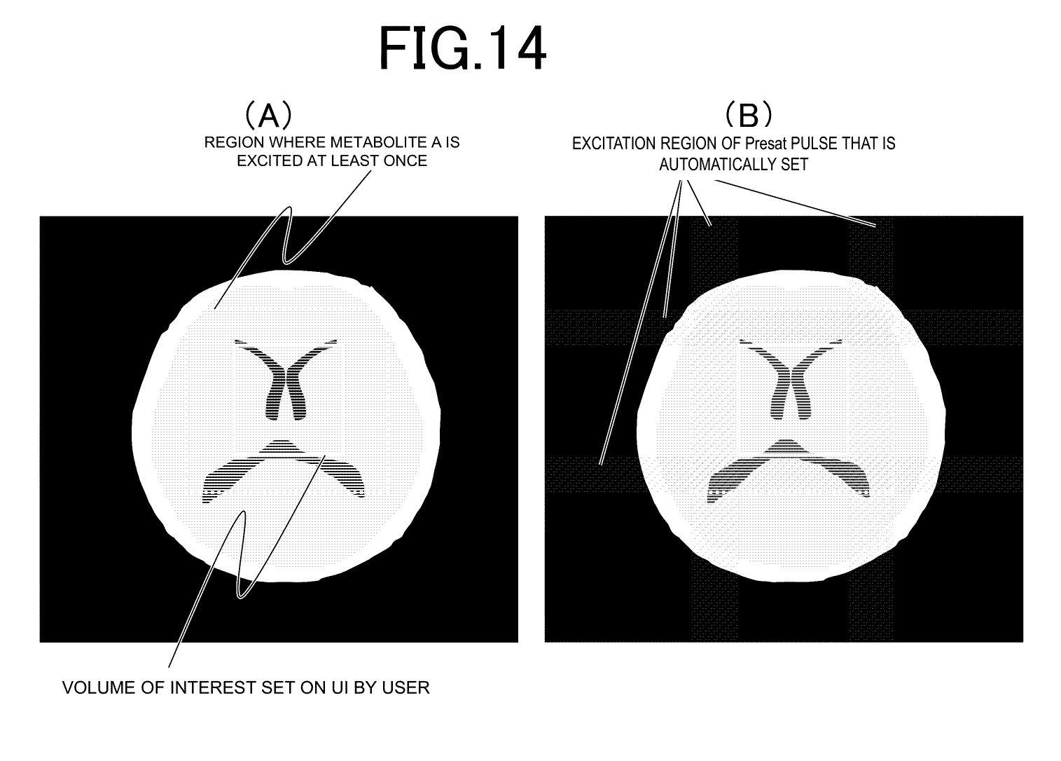

A fourth embodiment will be described with reference to FIGS. 13 and 14. The fourth embodiment is the same as the first embodiment up to the setting of a volume of interest in step S102, but is different from the first embodiment in that a Presat pulse is automatically set in step S503 thereafter. Subsequent steps are the same as those in the first embodiment. Hereinafter, details of step S503 and the reason why step S503 is provided will briefly be described. In addition, FIG. 14 is a monitoring region set by the user and an image that is displayed on the display 71 so that the positional relationship between the monitoring region and a region, which is excited by the Presat pulse and suppresses the generation of the NMR signal, can be seen. FIG. 14(A) shows a monitoring region set by the user and a region where the metabolite A specified outside the monitoring region is excited. FIG. 14(B) is an image created by the CPU 80 so that the positional relationship between a monitoring region set by the user and an excitation region by the Presat pulse, which is automatically calculated for the monitoring region, can be seen. This image is displayed on the display 71.

A metabolite having a resonance frequency farthest from the irradiation frequency is also considered herein. Here, the metabolite A is set as an example. When the third embodiment is performed, as shown in FIG. 14(A), signals from the outside of a volume of interest that the user desires are also measured. Therefore, in the present embodiment, regions where the metabolite A is unnecessarily excited outside the volume of interest are calculated, and the Presat pulse is automatically set so that excitation regions of the Presat pulse overlap these regions as shown in FIG. 14(B). The regions of the Presat pulse may not be displayed on the user interface, for example, on the display 71. Therefore, since the user can measure the signals of all metabolites included in the volume of interest without shortage, it is possible to reduce contamination signals from the outside of the volume of interest that are generated from a specific direction and then sufficiently suppress signals generated from the outside of the volume of interest by the Presat pulse.

Through the first to fourth embodiments, the magnetic resonance measuring apparatus of the present invention can acquire the spectral data with the reduced influence of contamination signals from the outside of the volume of interest. In addition, it is possible to prevent a reduction in the amount of signals within the setting region due to the excitation position shift. In addition, it is possible to suppress unnecessary signals from the outside of the setting region without a burden on the user.

While the embodiments of the present invention have been described, it is needless to say that the present invention may be applied to all excitation slice cross-sections of three axes of the X axis to the Z axis in these embodiments. However, a large effect can also be obtained even if the present invention is applied to the excitation slice cross-section of any one axis selected. In addition, it is needless to say that there is an effect even if the present invention is applied to the excitation slice cross-sections of any two axes. In the actual object, it is rare that metabolites considered to be a target are present in all directions. For this reason, an axis to apply the present invention or a range to apply the present invention may be selected corresponding to the state of the actual object. In this manner, it is possible to obtain the large effect.

### REFERENCE SIGNS LIST

1: object
10: sequencer
20: operating unit
21: pointing device (trackball or mouse)
22: keyboard
30: gradient magnetic field generation system
32: gradient magnetic field coil
34: static magnetic field generation magnet
36: gradient magnetic field power supply
40: static magnetic field generation system
50: signal transmission system
51: modulator
52: high frequency amplifier
53: high frequency coil on transmission side
54: high frequency oscillator
60: signal receiving system
61: A/D converter
62: quadrature phase detector
63: high frequency coil on receiving side
64: signal amplifier
70: control information processing system
71: display
72: optical disk
73: magnetic disk
74: ROM
75: RAM
80: central processing unit (CPU)
100: MRI apparatus
210: parameter setting unit
211: parameter input display section
212: position input display section
213: parameter calculating section
220: imaging unit
230: image reconstruction unit
240: display processing unit

1. A magnetic resonance imaging apparatus, comprising:
a static magnetic field generation unit configured to generate a uniform static magnetic field for an object;
a gradient magnetic field generation unit configured to generate a gradient magnetic field for the object;
a high-frequency pulse generation unit configured to generate a high-frequency pulse to be emitted to the object;
an echo signal receiving unit configured to receive an echo signal from the object; and
a control information processing unit configured to measure a state of a metabolite based on the received echo signal and a control the static magnetic field generation unit, the gradient magnetic field generation unit, or the high-frequency pulse generation unit,
wherein the control information processing unit acquires a first echo signal generated from the object based on a gradient magnetic field having one polarity generated by the gradient magnetic field generation unit, acquires a second echo signal generated from the object based on a gradient magnetic field having the other polarity, which is a polarity opposite to the one polarity, generated by the gradient magnetic field generation unit, and creates information indicating the state of the metabolite using both of the first and second echo signals.

2. The magnetic resonance imaging apparatus according to claim 1,
wherein an operating unit and a display are provided, the control information processing unit displays an input image for setting a volume of interest in the object on the display, and acquires a volume of interest in at least one direction of three directions perpendicular to each other through the operating unit,
the gradient magnetic field generation unit generates a gradient magnetic field for exciting the acquired volume of interest in one direction, and
the display displays the information indicating the state of the metabolite created by the control information processing unit.

3. The magnetic resonance imaging apparatus according to claim 2,
wherein the control information processing unit calculates a value by averaging both of the first and second echo signals, and creates the information indicating the state of the metabolite using the average value.

4. The magnetic resonance imaging apparatus according to claim 2,
wherein, when a metabolite is set and a volume of interest in at least one direction of three directions perpendicular to each other is set, the control information processing unit determines an excitation region by performing a calculation based on the set metabolite and the input volume of interest such that an excitation region where an excitation region of the set metabolite in a state in which the gradient magnetic field generation unit generates a gradient magnetic field with the one polarity and an excitation region of set metabolite in a state in which the gradient magnetic field generation unit generates a gradient magnetic field with the other polarity, which is a polarity opposite to the one polarity, overlap each other becomes the set volume of interest.

5. The magnetic resonance imaging apparatus according to claim 2,
wherein, when a metabolite is set and a volume of interest at least one direction of three directions perpendicular to each other is set, the control information processing unit determines an excitation region of the set metabolite, which is located outside the set volume of interest, by calculation, and applies a saturation pulse to the excitation region determined by the calculation.

6. The magnetic resonance imaging apparatus according to claim 2,
wherein the control information processing unit determines the excitation region by performing a calculation such that an excitation region where an excitation region of the set metabolite in a state in which the gradient magnetic field generation unit generates a gradient magnetic field with the one polarity and an excitation region of the set metabolite in a state in which the gradient magnetic field generation unit generates a gradient magnetic field with the other polarity, which is a polarity opposite to the one polarity, overlap each other becomes the set volume of interest in at least one direction of the three directions perpendicular to each other.

7. The magnetic resonance imaging apparatus according to claim 1,
wherein the control information processing unit creates a graph of the metabolite using both of the first and second echo signals.

8. The magnetic resonance imaging apparatus according to claim 1,
wherein the gradient magnetic field generation unit generates a gradient magnetic field having one polarity and a gradient magnetic field having the other polarity, which is a polarity opposite to the one polarity, in each direction of three directions perpendicular to each other, and
the control information processing unit acquires a volume of interest in each direction of the three directions perpendicular to each other, acquires both of the first and second echo signals in each direction of the three directions perpendicular to each other, and creates the information indicating the state of the metabolite using both of the first and second echo signals in all of the three directions perpendicular to each other.

9. The magnetic resonance imaging apparatus according to claim 1,
wherein the gradient magnetic field generation unit generates a gradient magnetic field having one polarity and a gradient magnetic field having the other polarity, which is a polarity opposite to the one polarity, in a direction of each of three axes of X, Y, and Z axes, and
the control information processing unit acquires a volume of interest in a direction of each of the three axes of the X, Y, and Z axes, acquires both of the first and second echo signals in a direction of each of the three axes of the X, Y, and Z axes, and creates the information indicating the state of the metabolite using both of the first and second echo signals in all of the three axes of the X, Y, and Z axes.

10. The magnetic resonance imaging apparatus according to claim 1,
wherein the gradient magnetic field generation unit generates a slice selection gradient magnetic field having one polarity and a slice selection gradient magnetic field having the other polarity, which is a polarity opposite to the one polarity, as the gradient magnetic fields,
the control information processing unit acquires the first echo signal based on the slice selection gradient magnetic field having the one polarity generated by the gradient magnetic field generation unit, acquires the second echo signal based on the slice selection gradient magnetic field having the other polarity generated by the gradient magnetic field generation unit, and creates the information indicating the state of the metabolite using both of the first and second echo signals.

11. The magnetic resonance imaging apparatus according to claim 1,
wherein the gradient magnetic field generation unit generates the gradient magnetic field having the one polarity and the gradient magnetic field having the other polarity alternately, and
the control information processing unit acquires the first and second echo signals alternately by a number designated in advance by acquiring the first echo signal in a state in which the gradient magnetic field generation unit generates the gradient magnetic field with the one polarity and acquiring the second echo signal in a state in which the gradient magnetic field generation unit generates the gradient magnetic field with the other polarity, and creates the information indicating the state of the metabolite using both of the acquired first and second echo signals.

12. The magnetic resonance imaging apparatus according to claim 1,
wherein the gradient magnetic field generation unit generates the gradient magnetic field having the one polarity repeatedly by a number designated in advance and generates the gradient magnetic field having the other polarity repeatedly by a number designated in advance, and
the control information processing unit acquires the first echo signal, which is generated according to the repetition of the generation of the gradient magnetic field having the one polarity by the gradient magnetic field generation unit, continuously and repeatedly by the number designated in advance, acquires the second echo signal, which is generated according to the repetition of the generation of the gradient magnetic field having the other polarity by the gradient magnetic field generation unit, continuously and repeatedly by the number designated in advance, and creates the information indicating the state of the metabolite using both of the acquired first and second echo signals.

13. The magnetic resonance imaging apparatus according to claim 1,
wherein the control information processing unit acquires the first echo signal, which is generated based on the gradient magnetic field having the one polarity generated by the gradient magnetic field generation unit, and the second echo signal, which is generated based on the gradient magnetic field having the other polarity generated by the gradient magnetic field generation unit, by the same number, and creates the information indicating the state of the metabolite using both of the acquired first and second echo signals.

14. The magnetic resonance imaging apparatus according to claim 2,
wherein, when an input of a volume of interest and an input of a setting of an excitation region are received, the control information processing unit creates an image showing a positional relationship between the volume of interest and the excitation region based on the inputs, and
the display displays the image created by the control information processing unit.

15. A measurement method of a magnetic resonance imaging apparatus, comprising:
a first step of generating a gradient magnetic field with one polarity for an object in a uniform static magnetic field and then acquiring a first echo signal generated by the object;
a second step of generating a gradient magnetic field with a polarity opposite to the one polarity and then acquiring a second echo signal generated by the object; and
a third step of creating information indicating a state of a metabolite using both of the first and second echo signals.
